(19) United States
(12) Patent Application Publication
(10) Pub. No.: US 2020/0395110 A1
(43) Pub. Date: Dec. 17, 2020

(54) METHOD AND SYSTEM FOR GENERATING STANDARDIZED CODES FROM ELECTRONIC RECORDS

(71) Applicant: NYM Health Ltd., Tel Aviv (IL)

(72) Inventor: Adam RIMON, Tel-Aviv (IL)

(73) Assignee: NYM Health Ltd., Tel Aviv (IL)

(21) Appl. No.: 16/677,217

(22) Filed: Nov. 7, 2019

Related U.S. Application Data
(60) Provisional application No. 62/861,551, filed on Jun. 14, 2019.

(51) Int. Cl. G16H 15/00; G16H 70/60; G16H 10/60; G06F 17/27

(52) U.S. Cl. CPC G16H 15/00; G06F 17/2785; G16H 10/60; G16H 70/60

(57) ABSTRACT

A system and method for generating standard codes for an input record. The method includes: receiving the input record, wherein the input record contains at least one sentence; performing a computational linguistic analysis on each of the least one sentence included the input record; constructing at least one narrative context the at least one sentence included in the input record; extracting at least one insight from the at least one narrative context; fetching at least one standard code for each of the at least one insight; and selecting at least one standard code at least one code best describing the extracted insight.

### CROSS-REFERENCE TO RELATED APPLICATIONS

This application claims the benefit of U.S. Provisional Application No. 62/861,551 filed on Jun. 14, 2019, the contents of which are hereby incorporated by reference.

### TECHNICAL FIELD

The present disclosure relates generally to an automated process of generating standard codes from textual descriptions, and, more specifically, to the generation of standard codes from electronic records.

### BACKGROUND

The business aspects of the medical field are complex and have many facets. In particular, in the presence of insurance companies that provide coverage to patients, there is a need to convert medical records into a series of codes that reflect the case at hand and that allow determination of the cost of all aspects resulting from the report provided in the medical record. Originally, the coding was done manually. However, as the number of codes increases, it has become impossible to manage the process which is error prone and difficult to monitor and invites fraud and inefficient use of available funds.

Various prior art solutions exist for the semi- or fully-automatic generation of evaluations and management schemes for medical codes, also referred to as E/M codes or E&M codes. These are based on complex sets of rules which may change from time-to-time and that are difficult to follow. Moreover, the rules may provide a result even if there is an error in the input data, inconsistencies between the codes generated and the actual description of the case, or other errors. It should be further noted that E/M codes are an example of the codes that may be used, and other codes may further be necessary for precise determination of the case at hand and any reimbursements due.

The possibility of errors is endlessly high in the entire revenue management cycle. For example coding may be inaccurate at the practitioner's side. As a result selection of wrong codes or sub optimal codes for a particular, identified condition may occur. The costs of these errors to the practitioners, patients, and insurance companies are significant. State-of-the-art solutions are designed for strict handling and, therefore, many charts are flagged for manual handling, which is labor intensive, time consuming, and is not free of errors either. A particular problem is the delay in denials of coverage by the insurance companies of coverage, which impacts both practitioners and patients. Another challenge has to do with the fact that codes evolve and are added and omitted as may be deemed necessary. There is a need to effectively track these changes and act accordingly. Moreover, there is a need, under the guidance, to be audit-ready so that compliance with the laws and regulations can be demonstrated at any time. All of these needs present multiple technical challenges that require resolution, preferably by reducing the human interaction to as few as possible edge cases.

Therefore, in view of the shortcomings of prior art approaches, it would be advantageous to provide an efficient solution for resolving the generation of codes from medical records. Furthermore, if would be beneficial to provide an audit chart that allows the audit of the process of the code generation.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “certain embodiments” may be used herein to refer to a single embodiment or multiple embodiments of the disclosure.

Certain embodiments disclosed herein include a method for generating standard codes for an input record. The method comprises the steps of receiving the input record, wherein the input record contains at least one sentence; performing a computational linguistic analysis on each of the at least one sentence included the input record; constructing at least one narrative context from the at least one sentence included in the input record; extracting at least one insight from the at least one narrative context; fetching at least one standard code for each of the at least one insight; and selecting at least one standard code best describing the extracted insight.

Certain embodiments disclosed herein also include a non-transitory computer readable medium having stored thereon instructions for causing a processing circuitry to perform a process for generating standard codes for an input record. The process comprises the steps of: receiving the input record, wherein the input record contains at least one sentence; performing a computational linguistic analysis on each of the least one sentence included the input record; constructing at least one narrative context from the at least one sentence included in the input record; extracting at least one insight from the at least one narrative context; fetching at least one standard code for each of the at least one insight; and selecting at least one standard code best describing the extracted insight.

In addition, certain embodiments disclosed herein include a system for generating standard codes for an input record. The system comprises a processing circuitry; and a memory, the memory containing instructions that, when executed by the processing circuitry, configure the system to receive the input record, wherein the input record contains at least one sentence; perform a computational linguistic analysis on each of the least one sentence included the input record; construct at least one narrative context from the at least one sentence included in the input record; extract at least one insight from the at least one narrative context; fetch at least one standard code for each of the at least one insight; and select at least one standard code best describing the extracted insight.

### BRIEF DESCRIPTION OF THE DRAWINGS

The subject matter that is regarded as the disclosure is particularly pointed out and distinctly claimed in the claims at the conclusion of the specification. The foregoing and other objects, features, and advantages of the invention will be apparent from the following detailed description taken in conjunction with the accompanying drawings.

FIG. 1 is a block diagram of a system for standard code generation based on an electronic record according to an embodiment.

FIG. 2 is a flowchart for generating standardized medical codes from electronic medical records according an embodiment.

FIG. 3 is an example of a sentence analyzed and identified as a valid sentence according to an embodiment.

FIG. 4 is another example of a sentence analyzed and identified as an invalid sentence according to an embodiment.

FIG. 5 is another example of sentences analyzed for the construction of a medical narrative across sentences according to an embodiment.

FIG. 6 is another example of sentences analyzed for the extraction of insights according to an embodiment.

FIG. 7 is a flowchart depicting for coding standard codes based on insights provided by a semantic engine according to an embodiment.

FIG. 8 illustrates an example coding based on insights according to an embodiment.

FIG. 9 is a screenshot of an audit trail chart generated according to an embodiment.

### DETAILED DESCRIPTION

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views. Moreover, some statements may apply to some inventive features but not to others. In general, unless otherwise indicated, singular elements may be in plural and vice versa with no loss of generality. In the drawings, like numerals refer to like parts through several views.

Some example embodiments include a system and method for processing medical records to output at least medical codes for each medical record. The system is configured to parse the text in the medical record and output valid structures for the purpose of coding, alerting of problem cases. The system is further configured to analyze sentences to accurately determine one or more standard codes, such as evaluation and management medical codes (EMCs), diagnosis codes or procedure codes, for the medical record thereby providing a fully autonomous coding. In addition, an audit trail chart may be generated for the standard codes. This allows an inspection of the process that led to the generation of the particular standard codes.

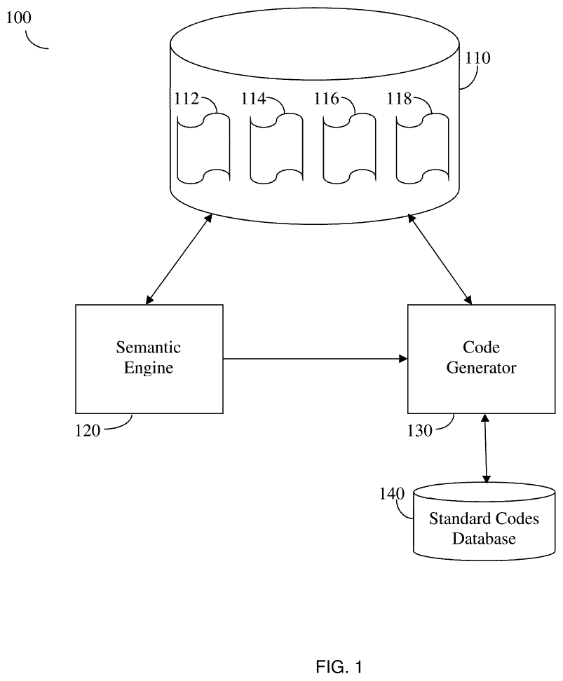

FIG. 1 is an example block diagram of a system 100 for standard code generation based on at least an input record according to an embodiment. The system 100 includes a first database 110 that contains therein one or more input records 112, for example medical records (MRs). A semantic engine (SE) 120 is communicatively connected to the first database 110 and configured to receive therefrom at least an input record 112. The SE 120 is configured to parse the text in the provided input record 112 to identify and capture every aspect of the input record 112.

For example, in the case of a medical record, every clinical aspect is identified and captured, the context understood, and relevance established. For example, an observation of an abdominal pain and a determination of an inflamed appendix can be associated. However, it is also possible that there are logical disconnects between the semantic analysis of the text and the relevancy. A symptom may be part of the record but not actually relevant to the particular case. In some cases, these should be flagged as errors and should be, in others, simply ignored as irrelevant. Therefore, the SE 120 is further configured to update an error report (ER) 114 in the first database 110 when such semantic inconsistencies are detected. The SE 120 is further configured to understand the meaning of a sentence using computational linguistics for words within a sentence and sentence structure.

The SE 120 is further configured to reconstruct the narrative of the sentences, for example a medical narrative, which is performed within and across separate sentences. This allows for the determination of the specific context of narrative. Thereafter, insights are extracted, which involves identifying the likes of speakers, entities, locations, as well as other specific features, including, for example in the medical field, anatomy, and, therefrom, detecting and potentially solving ambiguities. When this cannot be done, an error report (ER) 114 may be generated and stored in the first database 110.

It should be readily appreciated that the SE 120 and/or CG 130 may be implemented as processing circuitry coupled to a memory, the memory containing therein instructions that when executed by the processing circuitry perform the described functions of the SE 120 and/or CG 130. The processing circuitry may be realized as one or more hardware logic components and circuits. For example, and without limitation, illustrative types of hardware logic components that can be used include a central processing unit (CPU) field, programmable gate arrays (FPGAs), application-specific integrated circuits (ASICs), application-specific standard products (ASSPs), system-on-a-chip systems (SOCs), graphics processing units (GPUs), an artificial intelligence processor, i.e., processors configured or designed to perform machine learning tasks and make predictions based on their learnings, general-purpose microprocessors, microcontrollers, digital signal processors (DSPs), and the like, or any other hardware logic components that can perform calculations or other manipulations of information.

Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the one or more processors, cause the processing circuitry to perform the various processes described herein. Illustrative types of memory may include volatile memory such as, but not limited to random access memory (RAM), or non-volatile memory (NVM), such as, but not limited to, flash memory.

The SE 120 is communicatively connected a coding generator (CG) 130 which is configured to generate standard codes based on insights including, for example, medical insights, provided from the SE 120. The CG 130 is further communicatively connected to a second database 140 containing therein standard codes including, for example, codes relating to medical fields, such as ICD10 & CPT guidelines. The CG 130 is further configured to select one or more of the possible codes that are the best fit for the particular insight such as, for example, a clinical insight, provided by the SE 120.

The selected standard codes are stored in a code report (CR) 116. One of ordinary skill in the art would readily appreciate that the CG 130 may be implemented in a variety of ways, including, but not limited to, a CPU communicatively coupled to a memory, the memory containing therein instructions that, when executed by the CPU, perform the described functions of the CG 130. In another embodiment, an AIE or an MLE may be used and, through an initial training, may be capable of performing the tasks of the CG 130 described herein. In one embodiment, if an error is found in the coding process performed by the CG 130, an error is logged into ER 114.

In yet another embodiment, an audit trail chart is generated for allowing the tracking of the standard code(s) selected back to the input record that caused the codes' generation. By tracking such steps, it is possible to audit the process and ensure its validity, as further explained herein.

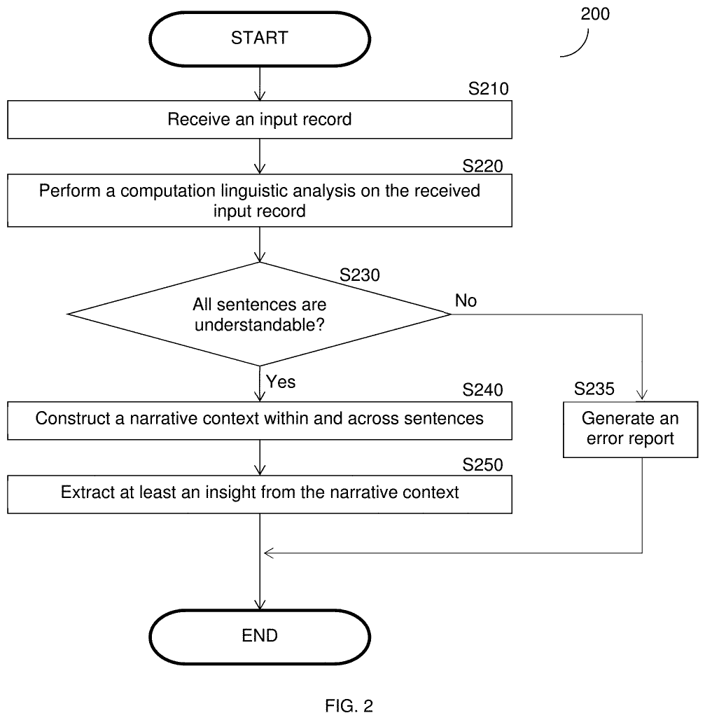

FIG. 2 is an example flowchart 200 for generating standardized medical codes from electronic medical records according an embodiment. The method may be performed by the SE 120 discussed above.

At S210 an input record, for example MR 112, is provided to a semantic engine, for example SE 120. The input record is an electronic medical record. At S220 a computational linguistic analysis on the received input record is performed.

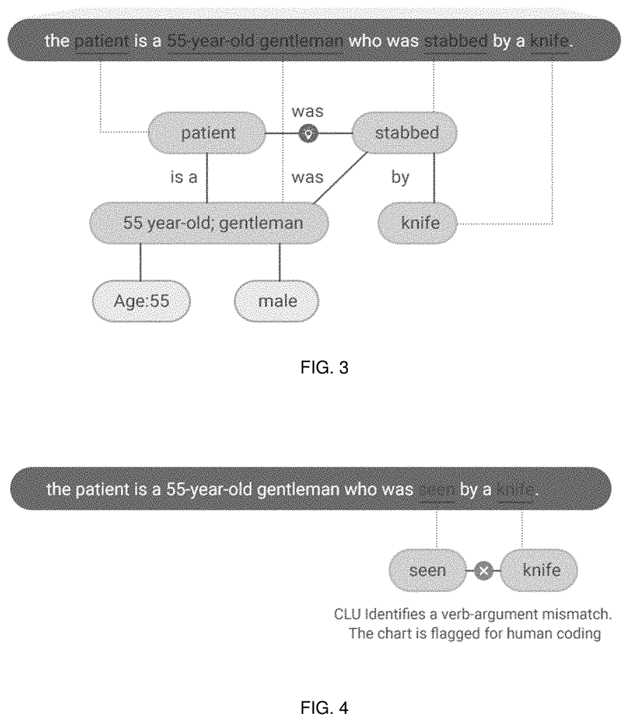

FIG. 3 shows an example of a computational linguistic analysis according to an embodiment. The first sentence “the patient is a 55-year-old gentleman who was stabbed by a knife” which is analyzed to be understood that the PATIENT was STABBED, that the PATIENT is a 55 YEAR-OLD; GENTLEMAN where his age is 55 and his gender is a MALE, and who was STABBED by a KNIFE. This sentence was an understood sentence and, therefore, no flagging is required. By contrast, a similar second sentence shown in FIG. 4, stating “the patient is a 55-year-old gentleman who was seen by a knife” would be flagged by this analysis as the language understanding performed according to an embodiment would identify the word SEEN and the word KNIFE as a verb mismatch.

Returning to FIG. 2, following the analysis at S220, at S230 it is checked whether no error has been found in sentences of the input record, and if so execution continues with S240; otherwise, execution continues with S235 where a report of the error, or errors, is generated, for example, by logging the error in the ER 114, after which execution terminates.

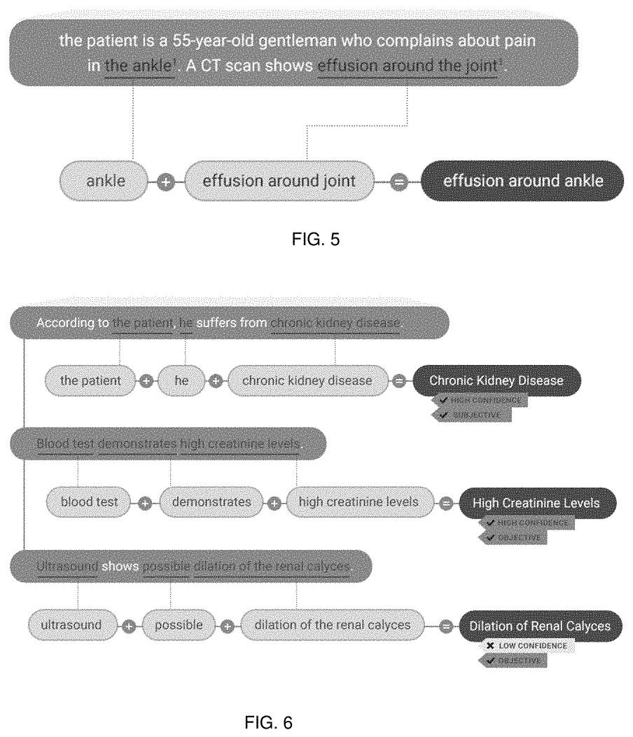

At S240, a construction of a narrative context is performed in a medical context making the narrative concisely understood from the textual description. This may be performed within or across sentences. Such a reconstruction is illustrated in FIG. 5. The third and fourth sentences “The patient is a 55-year-old gentleman who complains about pain in the ankle. A CT scan shows effusion around the joint.” An analysis would result in the identification of ANKLE, from third sentence, and EFFUSION AROUND JOINT, from the fourth sentence, as a narrative context, in this case, a medical narrative of an EFFUSION AROUND ANKLE.

At S250 insights are extracted based on the analyzed sentences. For each insight it is determined whether the finding is objective or subjective as well as the level of confidence in the particular insight. An example medical record is illustrated in FIG. 6 where a fifth, sixth and seventh sentences go through an insight extraction disclosed herein. The fifth sentence “According to the patient he suffers from chronic kidney disease.” Extracted therefrom are THE PATIENT, HE and CHRONIC KIDNEY DISEASE. The conclusion is that CHRONIC KIDNEY DISEASE satisfies for High Confidence but is Subjective as it is not backed by data such as lab tests or other objective kind of information. The sixth sentence reads “Blood test demonstrates high creatinine levels.”

There are three elements here: BLOOD TEST, DEMONSTRATES and HIGH CREATININE LEVELS which lead to the conclusion of HIGH CREATININE LEVELS as being in being High Confidence and Objective. The seventh sentence reads “Ultrasound shows possible dilation of the renal calyces.” This is analyzed based on ULTRASOUND, POSSIBLE and DIALATION OF THE RENAL CALYCES as DIALATION OF THE RENAL CALYCES being of Low Confidence but being also objective.

In one embodiment, S230 may be revised such that it is not required that all sentences have to be understandable. When it is determined that a particular sentence may be unimportant, such sentence is discarded from the list of the sentences required to be humanly understandable, i.e., a valid sentence in a spoken language. Such a determination may be made when implementing the SE 120 by an AI processor, or like processing capabilities, that is adapted to make such a determination. An unimportant sentence may be one that does not contain any valid or relevant medical information. In yet another embodiment, sentences are scored, rather than being identified, as in a binary way, as understood or not understood. In such a case, a threshold value may determine those cases which are not a clear cut case. In addition, in an embodiment, it is further possible to determine that an input record is not understandable if a certain number or percent of sentences, words, or combinations thereof are not understandable that is above a predetermined threshold.

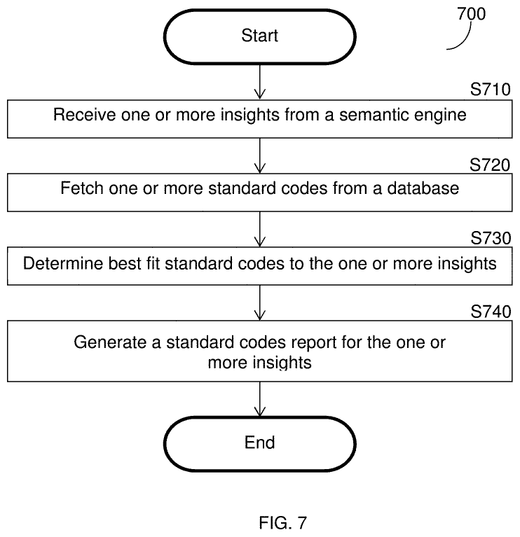

FIG. 7 shows an example flowchart 700 of coding into standard codes based on generated insights. In an embodiment, insights are generated by a semantic engine. At S710 insights are received or otherwise retrieved.

At S720, standard codes are fetched, for example standard codes are fetched or retrieved based on the insights. In an embodiment, the standard codes are fetched by the CG 130 (FIG. 1) from the second database 140 (FIG. 1). In an embodiment, the codes being fetched are standard medical code.

At S730 the best fitting standard codes are selected from standard codes fetched. Codes may include collections of characters. At S740 a code report and may be stored in a database (e.g. the first database).

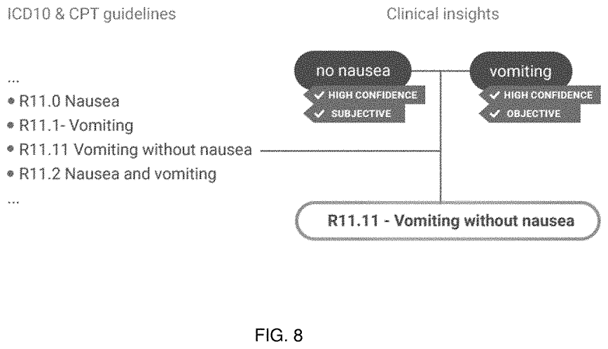

FIG. 8 illustrates an example coding based on insights according to an embodiment. The insights may be, for example, NO NAUSEA with High Confidence and being Subjective and VOMITING with High Confidence and being Objective. Therefore, codes for Nausea and Vomiting are fetched and include, but possibly not limited to: R11.0 Nausea; R11.1 Vomiting, R11.11 Vomiting without nausea; and R11.2 Nausea and vomiting. In this example, the standard code the R11.11 is selected as best fitting the insights provided. The standard codes in the medical field include EMCs, diagnosis codes, and procedure codes. EMCs are codes for measuring risks of a medical case; diagnosis codes represent diseases, symptoms and reasons for admission; and, procedure codes represent procedures and administrative resources invested in the medical case.

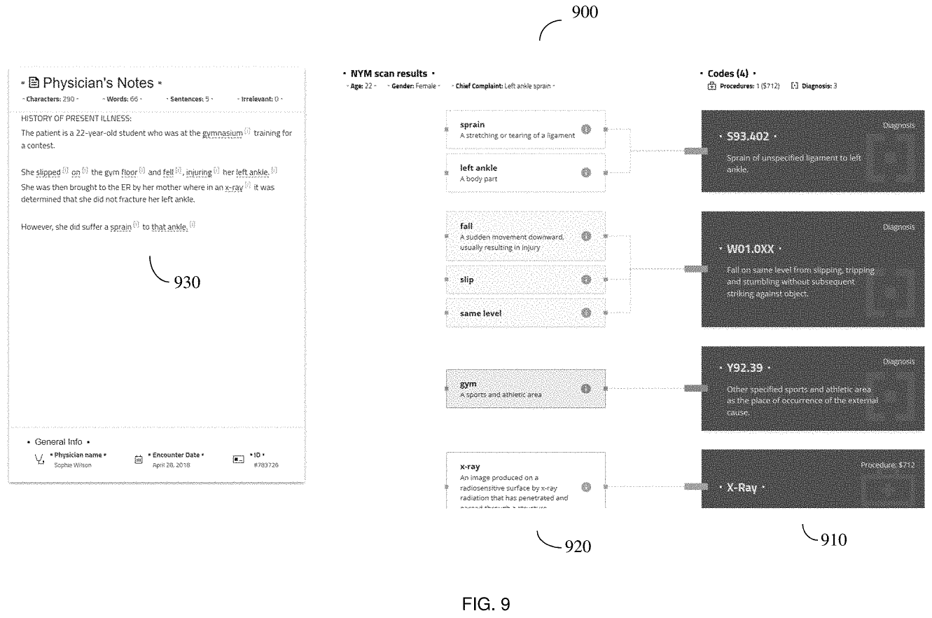

FIG. 9 is an example screenshot demonstrating of an audit trail report (ATC) 900 according to an embodiment. The ATC 900 (also shown in FIG. 1 as ATC 118) may be generated by SE 120 and/or CG 130 for the purpose of backtracking from the standard codes provided by the CG 130 back to the input record 112 that was used to provide them in the first place. By providing this report with its particular step present an auditor can easily establish the validity of the assignment of one or more standard codes based on a particular input record, for example a medical record.

In ATC 900, the codes selected can be tracked back, for each selected code 910, to the words 920 that triggered such a selection, and from the words back to the sentences 930 in which such words were found.

It should be appreciated that the system and methods described herein are not limited in scope to the field of medicine and may be equally applicable in other fields, for example, industrial fields such as aviation maintenance. Therefore, standard (or standardized) codes may include but are not limited to an observation code, a treatment code, a repair code, a part code, a component code, a medication code, a diagnosis code, a procedure code, an evaluation and management code, and the like.

The various embodiments disclosed herein may be implemented as hardware, firmware, software or any combination thereof. Moreover, the software is preferably implemented as an application program tangibly embodied on a program storage unit or computer readable medium. The application program may be uploaded to, and executed by, a machine comprising any suitable architecture. Preferably, the machine is implemented on a computer platform having hardware such as a processing unit (“CPU”), a memory, and input/output interfaces. The computer platform may also include an operating system and microinstruction code.

The various processes and functions described herein may be either part of the microinstruction code or part of the application program, or any combination thereof, which may be executed by a CPU that may be a combination of an array of processors. In addition, various other peripheral units may be connected to the computer platform such as an additional data storage unit and a printing unit. Furthermore, a non-transitory computer readable medium is any computer readable medium except for a transitory propagating signal.

The various embodiments disclosed herein can be implemented as hardware, firmware, software, or any combination thereof. Moreover, the software is preferably implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; A and B in combination; B and C in combination; A and C in combination; or A, B, and C in combination.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

What is claimed is:

1. A method for generating standard codes for an input record, comprising:
receiving the input record, wherein the input record contains at least one sentence;
performing a computational linguistic analysis on each of the least one sentence included the input record;
constructing at least one narrative context the at least one sentence included in the input record;
extracting at least one insight from the at least one narrative context;
fetching at least one standard code for each of the at least one insight; and
selecting at least one standard code best describing the extracted insight.

2. The method of claim 1, further comprising:
generating a code report containing the selected standard codes.

3. The method of claim 1, further comprises:
determining whether the input record is understandable; and
generating an error report when the input record is not understandable.

4. The method of claim 1, wherein the input record is at least medical record, and wherein the standard codes include medical codes.

5. The method of claim 4, wherein each of the medical codes are at least one of: an observation code, a treatment code, a repair code, a part code, a component code, a medication code, a diagnosis code, a procedure code, and an evaluation and management code.

6. The method of claim 5, wherein the extracted insight is related to: a medical condition.

7. The method of claim 1, further comprising:
generating an audit trail chart for tracking back from the at least one standard code to the input record; and
displaying the code generation path from the at least one standard code to the input record.

8. A non-transitory computer readable medium having stored thereon instructions for causing a processing circuitry to perform a process for generating standard codes for an input record, the process comprising:
receiving the input record, wherein the input record contains at least one sentence;
performing a computational linguistic analysis on each of the least one sentence included the input record;
constructing at least one narrative context the at least one sentence included in the input record;
extracting at least one insight from the at least a narrative context;
fetching at least one standard code for each of the at least one insight; and
selecting at least one standard code at least one code best describing the extracted insight.

9. A system for generating standard codes for an input record, comprising:
a processing circuitry; and
a memory, the memory containing instructions that, when executed by the processing circuitry, configure the system to:
receive the input record, wherein the input record contains at least one sentence;
perform a computational linguistic analysis on each of the least one sentence included the input record;
construct at least one narrative context the at least one sentence included in the input record;
extract at least one insight from the at least one narrative context;
fetch at least one standard code for each of the at least one insight; and
select at least one standard code at least one code best describing the extracted insight.

10. The system of claim 9, wherein the system is further configured to:
generate a code report containing the selected standard codes.

11. The system of claim 9, wherein the system is further configured to:
determine whether the input record is understandable; and
generate an error report when the input record is not understandable.

12. The system of claim 9, wherein the input record is at least medical record, and wherein the standard codes include medical codes.

13. The system of claim 12, wherein each of the medical codes are at least one of: an observation code, a treatment code, a repair code, a part code, a component code, a medication code, a diagnosis code, a procedure code, and an evaluation and management code.

14. The system of claim 13, wherein the extracted insight is related to: a medical condition.

15. The system of claim 9, wherein the system is further configured to:
generate an audit trail chart for tracking back from the at least one standard code to the input record; and
display the code generation path from the at least one standard code to the input record.
